(19) United States
(12) Patent Application Publication
(10) Pub. No.: US 2004/0033265 A1
(43) Pub. Date: Feb. 19, 2004

(54) Composite matrix containing chitosan derivatives and a process for making the same

(75) Inventors: Yuan Han Chen, Hsinchu (TW); Ming Hsi Yeh, Chiai (TW); Huey Min Lai, Hsinchu (TW)

Correspondence Address: BACON & THOMAS, PLLC, 625 SLATERS LANE, FOURTH FLOOR, ALEXANDRIA, VA, 22314

(73) Assignees: Industrial Technology Research Institute, Hsinchu (TW); Kuo-Cheng CHIU, Taipei (TW)

(21) Appl. No.: 10/329,712

(22) Filed: Dec. 27, 2002

(30) Foreign Application Priority Data
Dec. 31, 2001 (TW) 90133359

(51) Int. Cl. A61K 9/14

(52) U.S. Cl. 424/488; 514/55

(57) ABSTRACT

A method for preparing a composite matrix containing chitosan derivatives, comprising the steps of: (a) providing an anionic chitosan derivative solution (A); (b) providing a cationic polysaccharide solution (B); and (c) mixing solution (A) and solution (B) to form microcapsules. Metallic ion cross-linking agent and/or natural protein solution can be added optionally to adjust the mechanical strength of the shell and the interior physic state of the microcapsules.

### BACKGROUND OF THE INVENTION

1. Field of the Invention

The present invention relates to a composite matrix and a proparing process of the same, and, more particularly, to a composite matrix containing chitosan derivatives and process for preparing it.

2. Description of Related Art

Recently, for improving the drug delivering efficiency, increasing the routes to pass the bioactive components (including cells), and mitigating the immuno-rejection, scientists spends lots of efforts on searching or developing new materials or methods to deliver the drugs or bioactive materials via microcapsules. Among these developed materials, chitosan derivatives, which have cellulose-like straight-chain-backbone polymer structure is thought as a good natural polysaccharide suitable for drug delivery and encapsulating (or culturing) cells.

So far, alginate or methylpectin are popular conventional materials used for the formation of microcapsule matrix. Since the alginate or methylpectin carry charges and attract counter ions, microcapsules will form in the solution. The microcapsules formed by alginate or methylpectin can be further enhanced by the coating of polylysine. On the other hand, various methods are developed for preparing microcapsules based on these polysaccharide materials. These methods include physical methods, chemical methods, and spray dry mechanical methods.

However, the bioactive properties of conventional materials are inferior to chitosan derivatives. Chitosan and its derivatives have improved bio-adhesion and DNA delivery function since there are multiple bioactive functional groups attached on the backbone of chitosan or its derivatives. They also can be easily modified owing to their multiple functional groups on the backbones. The advantages of the multiple functional groups of chitosan is disclosed in U.S. Pat Nos. 4,803,168, 4,744,933, 5,089,272, 6,228,291 and 6,165,503, which show that chitosan is an improved material for microcapsule matrix. The microcapsules disclosed in these patents are consistent of chitosan carrying positive charge and alginate carrying negative charge.

On the other hand, after investigation, we found that the anionic chitosan derivatives, N, O-carboxymethylhcitosan (NOCC); can be used for the formation of microcapsules. NOCC could replace the conventional negative-charged alginate or methylpectin to form microcapsules. Furthermore, NOCC, which could also maintain moisture well, has been used for preparing hydrogel system. It suggests that NOCC can further preserve the interior liquid of the microcapsules, provide a good environment for culturing cells and maintain the activities of the drugs or bioactive materials, especially for improving the bioadhesiveness properties of mucosa cells and DNAs. So far, there is no research report for teaching the micro-encapsulation of NOCC and for improving the mechanical strength of such microcapsules. Therefore, it is desirable to provide a composite matrix containing chitosan derivatives to mitigate and/or obviate the aforementioned problems.

### SUMMARY OF THE INVENTION

The object of the present invention is to provide a method for preparing a composite matrix containing chitosan and its derivatives through the assistance of the attractive force between cationic and anionic polymers containing chitosan derivatives to form microcapsules.

Another object of the present invention is to provide a new microcapsule matrix formed by anionic chitosan derivative solution and cationic polysaccharide solution to improve the bio-degradability, biocompatibilities, bioadhesiveness, and the stabilities of the microcapsules. Said microcapsule matrix is also suitable for drug delivery, encapsulating or culturing cells.

To achieve the object, the method for preparing a composite matrix containing chitosan derivatives of the present invention comprising the steps of providing a solution (A) of an anionic chitosan or its derivatives; providing a solution (B) of a cationic polysaccharide; and mixing said solution (A) and said solution (B) together to form microcapsules.

Other objects, advantages, and novel features of the invention will become more apparent from the following detailed description when taken in conjunction with the accompanying drawings.

### DETAILED DESCRIPTION OF THE PREFERRED EMBODIMENT

The method of the present invention employs the attractive force between cationic and anionic polymer confining chitosan derivatives to form microcapsules. The anion donor is chitosan derivatives, and preferably, the anion donor is NOCC and its pharmaceutically acceptable salts. The cation donor is polysaccharide, and preferably, the cation donor is chitosan or chitosan derivatives dissolved in an acidic solution. The pH of said acidic solution of the present invention ranges from 2.0 to 6.0. Preferably, the acidic solution illustrated above is acetic acid solution, lactic acid solution, citric acid solution and diluted hydrochloric acid solution.

The micro-encapsulation process of the present invention comprises mixing the anionic chitosan derivative solution and the cationic polysaccharide solution together. The weight ratio of these two anionic chitosan and the cationic polysaccharide is not limited. Said mixing step could be achieved by adding one solution into the other stirring solution dropwise. The diameter of the microcapsule could be adjusted by controlling the size of the droplet. The stirring rate ranges from 100 rpm to 10,000 rpm; For improving the mechanical strength of the microcapsules' shell, said process can selectively comprise adding divalent or polyvalent metal ions to said solution for adjusting the mechanical strength of the microcapsules' shell. Preferably, the metal ion is calcium, barium, or chromium. The concentration of said divalent or polyvalent metal ions ranges from, 0 to 75 wt %. Furthermore, for meeting the various requirement of elasticity, moisture maintenance, and pharmaceutical function, a natural protein could be added optionally. Said natural protein could be added into anionic chitosan derivatives solution and/or cationic polysaccharide solution. The natural protein of the present invention is preferably selected from the natural hydrophilic protein group consisting of gelatin, collagen, fibrin, fibronectin, and elastin.

For encapsulating cells or drugs, the anionic chitosan derivative solution is first mixed with cells or drugs followed by adding cationic polysaccharide solution to form microcapsules. The other method is adding the cells or drugs to said cationic polysaccharide solution following by adding anionic chitosan derivative solution.

### EXAMPLE 1

NOCC Solution Dropped into Calcium Solution

2 wt % NOCC solution is added. into 1-5 M of stirring calcium chloride solution dropwise. The NOCC converses to form microcapsules immediately when it contacts the calcium chloride solution. The diameter of the microcapsules could be adjusted by controlling the size of the droplet. The diameter of thus obtained microcapsules ranges from 8 mm to 0.2 mm. The shell of the microcapsules increases with soaking time and microcapsules with substantial core will form ultimately.

### EXAMPLE 2

NOCC Solution Dropped into Chitosan Solution

2 wt % NOCC solution is added into 1 wt % to 4 wt % of stirring chitosan solution dropwise. The NOCC converses to form microcapsules immediately when it contacts the chitosan solution. The diameter of the microcapsule could be adjusted by, controlling the size of the droplet. The diameter of thus obtained microcapsules ranges from 8 mm to 0.2 mm. The shell of the microcapsules increases with soaking time, and microcapsules with liquid interior will form ultimately.

### EXAMPLE 3

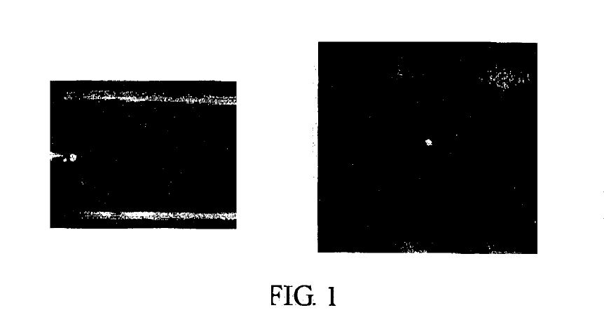

NOCC Solution Dropped into Mixture Containing Chitosan and Calcium Chloride Solution (FIG. 1)

2 wt % NOCC solution is dropped into the stirring mixture containing chitosan and calcium chloride solution. The mixture consists of 1 wt % to 4 wt % of chitosan dissolved in an acetic acid solution and 1-5 M of calcium chloride solution, wherein the weight ratio of chitosan to calcium ion is 3:1, 1:1 or 1:2. The NOCC converses to microcapsules immediately when it contacts the mixture. The diameter of the microcapsule could be adjusted by controlling size of the droplet. The diameter of thus obtained microcapsules ranges from 8 mm to 0.2 mm. The shell of the microcapsules increases with soaking time, and microcapsules with liquid interior will form ultimately.

### EXAMPLE 4

NOCC Solution Dropped into Mixture Containing Collagen and Calcium Chloride Solution

2 wt % NOCC solution is dropped into the stirring mixture containing collagen and calcium chloride solution. The mixture consists of 1 wt % collagen dissolved in 1 wt % acetic acid solution and 1-5 M of calcium chloride solution, wherein the weight ratio of chitosan to calcium ion is 9:1. The NOCC converses to microcapsules immediately when it contacts the mixture. The diameter of the microcapsule could be adjusted by controlling the size of the droplet. The diameter of thus obtained microcapsules ranges from 8 mm to 0.2 mm. The shell of the microcapsules increases with soaking time, and microcapsules with substantial core will form ultimately.

### EXAMPLE 5

NOCC Solution Dropped into Mixture Containing Chitosan, Collagen and Calcium Chloride Solution

2 wt % NOCC solution is dropped into the stirring mixture containing chitosan, collagen and calcium chloride solution. The mixture consists of 1 wt % to 4 wt % of chitosan dissolved in 1 wt % acetic acid solution, 1 wt % collagen dissolved in 1 wt % acetic acid solution, and 1-5 M of calcium chloride solution, wherein the weight ratio of chitosan to collagen to calcium ion is 6:1:3, 9:2:9, 3:1:6. The NOCC will converse to microcapsules immediately when it contacts the mixture. The diamneter of the microcapsule could be adjusted by controlling the size of the droplet. The diameter of thus obtained microcapsules ranges 8 mm to 0.2 mm. The shell of the microcapsules increases with soaking time, and microcapsules with liquid interior will form ultimately.

### EXAMPLE 6

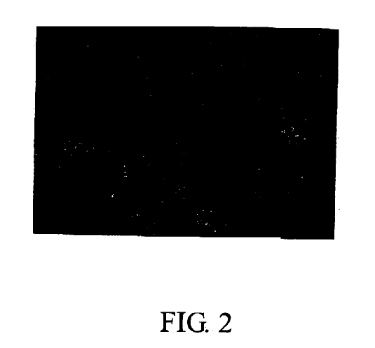

NOCC and Collagen Solution Dropped into Mixture Containing Chitosan and Calcium Solution (FIG. 2)

2 wt % NOCC solution and 1 wt % collagen dissolved into 1 wt % acetic acid solution are mixed well with a weight ratio of 9:1 or 4:1 and droped into the stirring mixture containing chitosan and calcium chloride solution. The mixture consists of 1 wt % to 4 wt % of chitosan dissolved in 1 wt % acetic acid solution and 1-5 M of calcium chloride solution, wherein the weight ratio of chitosan to calcium ion is 2:1, 1:1, or 1:2. The NOCC converses, to microcapsules immediately when it contacts the mixture. The diameter of the microcapsule could be adjusted by controlling the size of the droplet. The diameter of thus obtained microcapsules ranges from 8 mm to 0.2 mm. The shell of the microcapsules increases soaking time, and microcapsules with liquid interior will form ultimately.

The process of the present invention combines the shaping, coating and cross-linking in one step, which is much simpler than prior art. In addition, the thickness of the microcapsules shell can be adjusted by controlling the concentration of the counter ions and the cross-linking agent. The present invention discloses that NOCC can serve as microcapsule matrix, which provides an excellent environment suitable for cell culture and maintaining the biomaterial's activity.

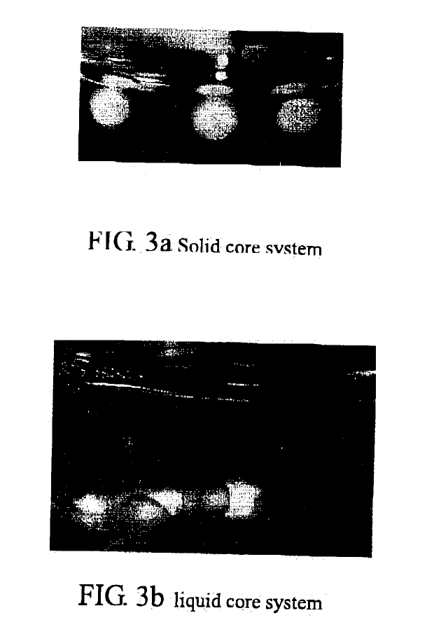

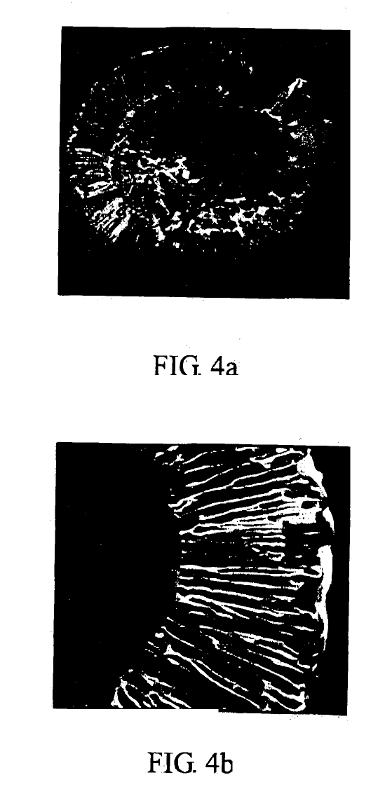

Since people skilled in this field know that the biomaterials containing chitosan derivatives exhibit special bio-adhesion and DNA delivery abilities, it can be realized that the matrix of the present invention, comprising both cationic chitosan derivatives and anionic chitosan derivatives, exhibits much better bio-adhesion and DNA delivery abilities than the prior arts did. In another aspect, this is the first time to disclose a new microencapsulation system, which is not disturbed by adding natural proteins with high biocompatibility. The natural protein added could improve the elasticity, moisture maintenance, and specific function of the microcapsules suitable for various applications. Additionally, the microcapsules of the present invention could be modified to exhibit more bio-functions. Moreover, the thickness, hardness and interior state could be adjusted by changing the condition of the manufacturing process to further control the released rate of the drug inside the microcapsules (FIG. 3a, b). Therefore, the microcapsules produced according the present invention are channel-like structure which is very suitable for drug delivery and tissue engineering application (FIG. 4a,b).

Although the present invention has been explained in relation to its preferred embodiment, it is to be understood that many other possible modifications and variations can be made without departings from the spirit and scope of the invention as hereinafter claimed.

What is claimed is:

1. A method for preparing a composite matrix containing chitosan or its derivatives, comprising following steps:
(a) providing a solution (A) of an anionic chitosan or its derivatives;
(b) providing a solution (B) of a cationic polysaccharide; and (c) mixing said solution (A) and said solution (B) together to form microcapsules.

2. The method as claimed in claim 1, further comprising adding a solution of divalent or polyvalent metal ions to the solution (B) in step (b) for adjusting the mechanical strength of the microcapsules' shell.

3. The method as claimed in claim 2, wherein at least one divalent or polyvalent metal ion is selected from the group consisting of calcium barium, and chromium.

4. The method as claimed in claim 2, wherein the concentration of said divalent or polyvalent metal ions ranges from 0 to 75 wt %.

5. The method as claimed in claim 1, further comprising adding natural protein solution to said solution (A) in step (a) and/or adding natural protein solution to said solution (B) in step (b).

6. The method as claimed in claim 5, wherein at least one natural protein is selected from the group consisting of gelatin, collagen, fibrin, fibronectin, and elastin.

7. The method as claimed in claim 5, wherein said the final concentration of said natural protein solution after mixing in step (c) is no more than 40 wt %.

8. The method as claimed in claim 1, wherein said anionic chitosan derivative in step (a) is N, O-carboxymethylchitosan (OCC).

9. The method as claimed in claim 1, wherein said solution of said cationic polysaccharide in step (b) is a solution of acidic polysaccharide or a solution of acidic chitosan derivative.

10. The method as claimed in claim 9, wherein the pH value of said solution of said acidic polysaccharide or acidic chitosan derivative ranges from 2.0 to 6.0.

11. The method as claimed in claim 9, wherein at least one said acidic solution is made from the acid selected from the group consisting of acetic acid solution, lactic acid solution, citric acid solution and diluted hydrochloric acid solution.

12. The method as claimed in claim 1, said step (c) further comprising a step for stirring the mixture with a stirring rate between 100 rpm and 10,000 rpm.

13. A composite matrix containing chitosan derivatives, which is produced by mixing an anionic chitosan derivative solution and a cationic polysaccharide solution.

14. The composite; matrix as claimed in claim 13, further comprising adding one or more divalent or polyvalent metal ions to said mixture for adjusting the mechanical strength of the microcapsules' shell; wherein at least one divalent or polyvalent metal is selected from the group consisting of calcium, barium, and chromium.

15. The composite matrix as claimed in claim 13, further comprising at least one natural protein solution.

16. The composite matrix as claimed in claim 15, wherein at least one natural protein is selected from the natural hydrophilic protein group consisting of gelatin, collagen, fibrin, fibronectin, and elastin.

17. The composite matrix as claimed in claim 13, wherein said anionic chitosan derivative in step (a) is N, O-carboxymethylchitosan (NOCC).

18. The composite matrix as claimed in claim 13, wherein said cationic polysaccharide is selected from acidic polysaccharide solution or acidic chitosan derivative solution.

19. The composite matrix as claimed in claim 18, wherein at least one acidic solution is selected from the group consisting of acetic acid solution, lactic acid solution, citric acid solution and diluted hydrochloric acid solution.

20. The composite matrix as claimed in claim 14, wherein the concentration of said divalent or polyvalent metallic coupling agent ranges from 0 to 75 wt %.
